(19) United States
(12) Patent Application Publication
(10) Pub. No.: US 2016/0068803 A1
(43) Pub. Date: Mar. 10, 2016

(54) Production of tailored PHA copolymers with methane and added co-substrates

(71) Applicant: The Board of Trustees of the Leland Stanford Junior University, Palo Alto, CA (US)

(72) Inventors: Craig S. Criddle, Redwood City, CA (US); Jaewook Myung, Stanford, CA (US)

(21) Appl. No.: 14/825,473

(22) Filed: Aug. 13, 2015

Related U.S. Application Data
(60) Provisional application No. 62/047,987, filed on Sep. 9, 2014.

(51) Int. Cl. C12N 1/20

(52) U.S. Cl. CPC C12N 1/20

(57) ABSTRACT

A method of producing polyhydroxyalkanoic acid (PHA)-producing biomass that includes using a first bioreactor for growth of methanotrophic biomass, flushing the methanotrophic biomass with a CH₄:O₂ mixture and providing nutrients needed for sustained cell division, removing a portion of the flushed biomass, where the remainder is retained in the first bioreactor as starter biomass for continuous cycles of cell replication, transferring the removed biomass to a second bioreactor, incubating the removed biomass in the second bioreactor with a CH₄:O₂ mixture or CH₃OH:O₂ mixture in the absence of sufficient nutrients for cell replication and in the presence of a co-substrate, and harvesting PHA-containing cells from the second bioreactor.

### CROSS-REFERENCE TO RELATED APPLICATIONS

This application claims priority from U.S. Provisional Patent Application 62/047,987 filed Sep. 9, 2014, which is incorporated herein by reference.

### FIELD OF THE INVENTION

The present invention relates generally to polyhydroxyalkanoic acid (PHA) production. More particularly, the invention relates to an efficient and cost effective method of PHA production using methane or methanol and to enable tailored synthesis of copolymers.

### BACKGROUND OF THE INVENTION

Polyhydroxyalkanoates (PHAs) are biodegradable, biocompatible and renewable bioplastics that could substitute for petrochemical-derived plastics in many applications, decreasing greenhouse gas emissions. Use of methane (CH₄) as a feedstock for PHA production can significantly decrease costs and environmental impacts. To date, however, production of PHA by obligate methanotrophs is limited to poly(3-hydroxybutyrate) (P3HB). Consistent production of P3HB ensures a uniform product, but limits flexibility in responding to market demands because P3HB has narrow melt processing windows and lacks flexibility. The market of CH₄-derived PHAs could be expanded by incorporating 3-hydroxyvalerate (3HV) or other monomers into the PHA polymer. Increasing 3HV content decreases melting temperature (Tₘ), glass transition temperature (T_{g}), crystallinity and water permeability, and increases impact strength and flexibility.

Many nutrient-limited heterotrophic bacteria incorporate 3HV units derived from odd carbon fatty acids, such as propionate or valerate, into PHAs, but obligate methanotrophs are unable to grow with fatty acids. They would thus not be expected to use odd carbon fatty acids as a source of 3HV units. Recent studies indicate that some methanotrophs in Methylocystis genus can utilize multi-carbon substrates for growth, but this capacity is limited to only a few strains.

What is needed is a method of synthesis of poly(3-hydroxybutyrate-co-3-hydroxyvalerate) (PHBV) in Methylocystis parvus OBBP and Methylosinus trichosporium OB3b.

### SUMMARY OF THE INVENTION

To address the needs in the art, a method of producing polyhydroxyalkanoic acid (PHA)-producing biomass is provided that includes using a first bioreactor for growth of methanotrophic biomass, flushing the methanotrophic biomass with a CH₄:O₂ mixture and providing nutrients needed for sustained cell division, removing a portion of the flushed biomass, where the remainder is retained in the first bioreactor as starter biomass for continuous cycles of cell replication, transferring the removed biomass to a second bioreactor, incubating the removed biomass in the second bioreactor with a CH₄:O₂ mixture or CH₃OH:O₂ mixture in the absence of sufficient nutrients for cell replication and in the presence of a co-substrate, and harvesting PHA-containing cells from the second bioreactor.

According to one aspect of the invention, the co-substrate includes fatty acids, where the fatty acids include valerate, ¹³C-carbonyl labeled-valerate, 3-hydroxybutyrate, 4-hydroxybutyrate, or propionate.

According to another aspect of the invention, the PHA-containing cells are copolymers of PHA that include poly(3-hydroxybutyric acid-co-3-hydroxyvaleric acid) (PHBV), block-poly-3-hydroxyhexanoate (PHB-b-PHHx) or poly(3-hydroxybutyrate-co-4-hydroxybutyrate) (P3HB4HB). In one aspect, the co-substrate for the poly(3-hydroxybutyric acid-co-3-hydroxyvaleric acid) (PHBV) includes 2-pentenoate. In another aspect, the co-substrate for the block-poly-3-hydroxyhexanoate (PHB-b-PHHx) includes hexanoate. In a further aspect, the co-substrate for the poly(3-hydroxybutyrate-co-4-hydroxybutyrate) (P3HB4HB) includes 4-hydroxybutyrate and gamma butyrolactone (GBL).

### BRIEF DESCRIPTION OF THE DRAWINGS

FIG. 1 shows 3HV fraction in resulting PHBV produced relative to initial valerate concentration, according to one aspect of the invention.

FIG. 2 shows the percent PHBV produced relative to initial valerate concentration, according to one aspect of the invention.

FIGS. 3A-3B show ¹³C-NMR spectra (125 MHz, CDCl₃) of PHBV copolymers produced, where PHBV copolymers were produced with naturally abundant valerate (FIG. 3A, top spectrum) and ¹³C-carbonyl labeled-valerate as co-substrates (FIG. 3B, bottom spectrum), and the numbering of the atoms is illustrated on a valerate-butyrate diad, according to one aspect of the invention.

FIG. 4 shows a flow diagram of a method of growing polyhydroxyalkanoic acid (PHA) producing bacteria under nitrogen-limited growth conditions, according to one aspect of the invention.

### DETAILED DESCRIPTION

This invention provides a method for use of methane as the primary substrate for growth of polyhydroxyalkanoic acid (PHA) producing methane-oxidizing bacteria where the PHA production phase can be modified through addition of co-substrates to enable production of co-polymers with desirable properties for a range of applications. According to one embodiment, methane is used to support replication of methanotrophic bacteria in a balanced growth phase, i.e., a phase in which sufficient major and minor nutrients are present to support cell division, where in addition to organic energy and carbon sources, bacteria require a number of other nutrients including nitrogen, phosphorus, sulfur, trace metals and salts. Absence of any of these components restrains cells from replication. This is followed by an unbalanced growth phase in which one or more nutrients is limiting, preventing further cell division. Under such conditions, 3-hydroxyacyl-CoA occurs through the synthesis of intracellular PHA granules, where 3-hydroxyvaleryl-CoA is supplied to produce PHBV copolymer. Further, P3HB homopolymer will be produced when only 3-hydroxybutyryl-CoA is present. Or it can generally be termed as. During this phase, both methane and co-substrates are added to enable production of customized PHA co-polymers, with variable side chain composition and/or variable number of carbon atoms in the polymer backbone. Such modifications can confer many useful properties, such as impact resistance, toughness, and flexibility.

While the methodology used for production of copolymers is observed in a wide range of bacteria, it is not obvious that methanotrophic bacteria, which are generally believed to be restricted to one carbon metabolism and use the stored polymer in a fashion different from other bacteria, would possess the capacity to produce granules of differing copolymer composition. Such a capacity has not been previously reported or observed in methanotrophic bacteria. Changes in the number of carbon atoms in the monomer side chains depends upon the number of carbon atoms in the substrates added during the polymer production phase: when this number is even, the side chains contain an odd number of carbon atoms; when it is odd, the side chains contain an even number of carbon atoms. For acetate, beta oxidation can promote incorporation of 3HB units via successive formation of acetyl CoA, acetoacetyl-CoA, 3-hydroxybutyryl-CoA, and ultimately 3HB monomers, with methyl side chains. It is anticipated that longer side chains may result through addition of longer alkanoates containing an even number of carbon atoms. For added alkanoates containing an odd number of carbon atoms, such as valerate, hydroxyacyl units may be added via beta oxidation, with formation of acyl-CoA, 2-enoyl-CoA, 3-hydroxyacyl-CoA, and incorporation of 3-hydroxyacyl units (resulting in side chains with an even number of carbon atoms, such as ethyl groups). For alkenoates, such as crotonate and 2-pentenoate, a possible pathway is thiolase-mediated formation of enoyl-CoA, hydratase-mediated formation of 3-hydroxyacyl-CoA, and PHA-synthase incorporation of 3-hydroxyacyl units. Changes in the number of carbon atoms in the backbone of the copolymer are achieved in one of two ways: (1) by addition of a hydroxyalkanoate co-substrate with the hydroxyl group located on the terminal carbon atom, or (2) by addition of an alkenenoic acid in which a double bond is located between the terminal and sub-terminal carbon atoms.

PHAs are of value for many applications, including packaging, toys, 3D printing, biocomposites, and many other applications. They are the fastest growing biopolymer market with a compound annual growth rate of 28% to 2018. This market is expected to be worth $3.7 billion by 2018

The current invention is the first discovery of a methodology for production of co-polymers, with methane as the primary substrate for biomass production. Typically, methane-oxidizing bacteria capable of PHA production produce only poly-3-hydroxybutyric acid (P3HB). P3HB is a relatively brittle polymer, unsuitable for many applications. By contrast, copolymers of P3HB, such as poly(3-hydroxybutyric acid-co-3-hydroxyvaleric acid) (PHBV), block-poly-3-hydroxyhexanoate (PHB-b-PHHx) or poly(3-hydroxybutyrate-co-4-hydroxybutyrate) (P3HB4HB), can be used in a wide range of applications.

The current invention may involve pure cultures of methanotrophs or enrichments, and may entail continuous or batch cultivation.

This method enables use of a cheap and abundant substrate to support growth and P3HB production, with selective addition of co-substrates to customize the co-polymer.

In another aspect of the invention, addition of propionate or valerate as co-substrates with CH₄ or methanol (CH₃OH) during the period of polymer formation enables synthesis of poly(3-hydroxybutyrate-co-3-hydroxyvalerate) (PHBV) in Methylocystis parvus OBBP. This finding is extended to Methylosinus trichosporium OB3b, another Type II obligate methanotroph.

Turning now to PHBV production, TABLE 1 summarizes PHBV production results for M. parvus OBBP. When 3HB was added as a co-substrate during the PHA production step (without nitrogen in the incubation medium) and CH₄ was also present, P3HB content increased from 50±5 to 60±5 wt %, indicating efficient incorporation of the 3HB monomer. The change in P3HB wt % that resulted was statistically significant (p-value=0.0352). When CH₃OH (2 g/L) was supplied in place of CH₄, the final P3HB content remained stable (59±4 wt %). 3HB is a product of the abiotic hydrolysis of PHAs, suggesting a route for abiotic-biotic recycling. These results also show that M. parvus OBBP possesses a mechanism for uptake of fatty acids, and that the mechanisms requires oxidation of CH₄ or CH₃OH. M. parvus OBBP does not grow on acetate, but acetate uptake has been reported for Methylocystis strain SB2 during cell division.

TABLE 1
PHBV production by M. parvus OBBP after 48-h incubation
with/without CH₄ and different co-substrates.

| | Presence | PHA | 3HV |
|---|---|---|---|
| Co-substrate | of CH₄? | content (wt %) | fraction (mol %) |
| None | Yes | 50 ± 5 | 0 |
| | No | 0 | — |
| 3-hydroxybutyrate | Yes | 60 ± 5 | 0 |
| (100 mg/L) | No | 0 | — |
| Propionate | Yes | 32 ± 4 | 8 ± 2 |
| (100 mg/L) | No | 0 | — |
| Valerate | Yes | 54 ± 3 | 22 ± 3 |
| (100 mg/L) | No | 0 | — |

When odd carbon number volatile fatty acids (propionate or valerate) were added as co-substrates, 3HV units were incorporated (TABLE 1). With 100 mg/L propionate, the 3HV fraction was 8±2 mol %, and the final PHA content was 32±4 wt %. The fraction of propionate incorporated into 3HV monomers relative to the total propionate taken up to by the cells ranged from 75-90%. Addition of 100 mg/L valerate did not significantly change PHA content (50±5 wt % without valerate; 54±3 wt % with valerate), but did increase the 3HV fraction from zero, in absence of valerate, to 22±3 mol % with added valerate. The fraction of valerate incorporated into 3HV units relative to the total valerate taken up by the cells ranged from 88-95%. When CH₃OH (2 g/L) was supplied in place of CH₄, the 3HV mol % remained stable (22±4%) as did the final wt % PHA (52±7%). For M. trichosporium OB3b very similar results were obtained: addition of 100 mg/L valerate did not significantly change PHA content (47±3 wt % PHA without valerate; 50±4 wt % with valerate), and the 3HV content increased from zero to 20±4 mol % 3HV.

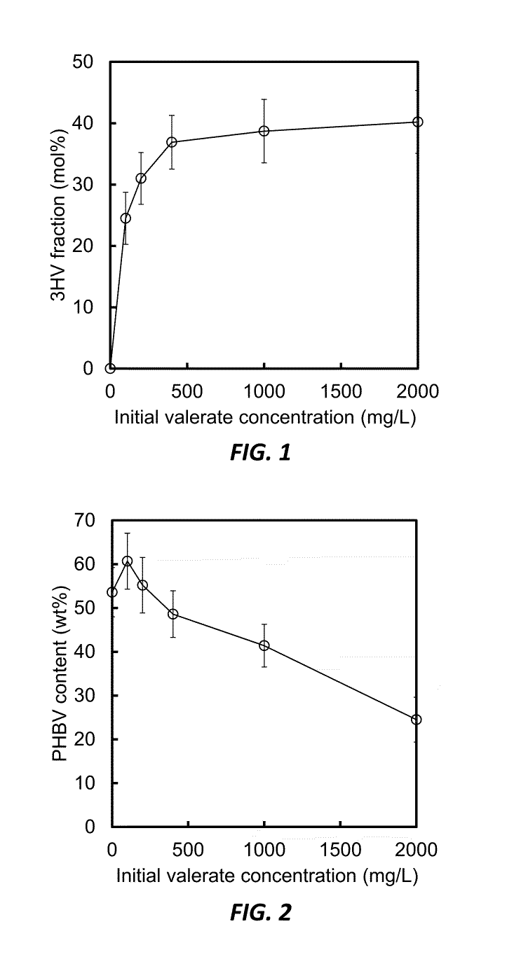

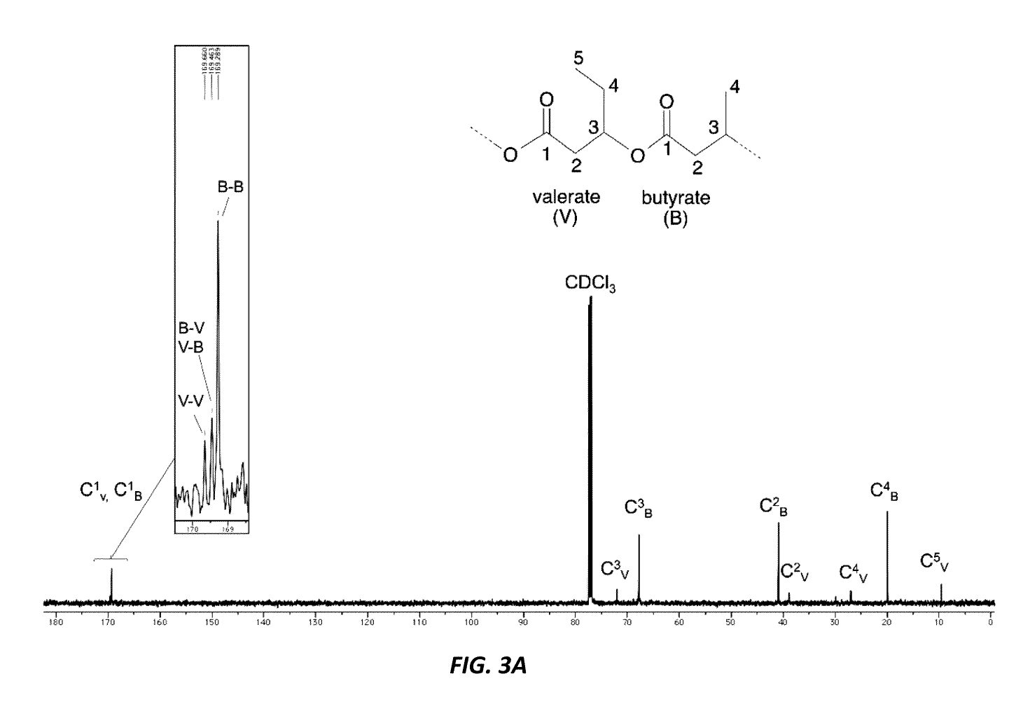

To further understand the effect of added co-substrate on reaction stoichiometry and kinetics, a range of valerate concentrations were added and the mol % 3HV was monitored (FIG. 1) and wt % PHA (FIG. 2). For added valerate levels <500 mg/L, the mol % 3HV of the PHA copolymer increased with increasing valerate concentrations. At higher levels, the 3HV fraction stabilized at ˜40%. At lower initial concentration of valerate (100 mg/L), the wt % PHA increased slightly, and at higher valerate concentrations, the wt % PHA decreased.

TABLE 2 illustrates molecular weight and molecular weight distributions (PDI=M_{w}/Mₙ) of P3HB and PHBV generated. These values are comparable to those of heterotrophic enrichments known in the art and to commercial P3HB and PHBV powders, but are more uniform, with higher molecular weights and lower PDI values.

TABLE 2
Peak molecular weights and polydispersity
indices (PDI) for extracted PHAs.

| Incubation condition | Polymer detected | Peak molecular weight | PDI |
|---|---|---|---|
| Methane alone | P3HB | 1.24E+06 | 1.67 |
| Methane + 100 | PHBV with 22 | 1.13E+06 | 1.88 |
| mg/L valerate | mol % 3HV | | |
| Methane + 400 | PHBV with 37 | 8.70E+05 | 2.23 |
| mg/L valerate | mol % 3HV | | |

Differential scanning calorimetry (DSC) analysis on PHA polymers produced with CH₄ alone had a peak melting temperature (Tₘ) of 178° C. and an onset glass transition temperature (T⁰_{g}) of 8° C., values typical of P3HB. The PHA polymers containing 3HV units had peak melting temperatures (Tₘ) of 150° C. (3HV fraction of 22 mol %) and 134° C. (3HV fraction of 37 mol %), and onset glass temperatures of (T⁰_{g}) of −2° C. and −6° C., values typical of PHBV. The presence of only one peak melting temperature is evidence that the PHA polymer produced with propionate or valerate is PHBV, and not a blend of P3HB and poly(3-hydroxyvalerate) (P3HV).

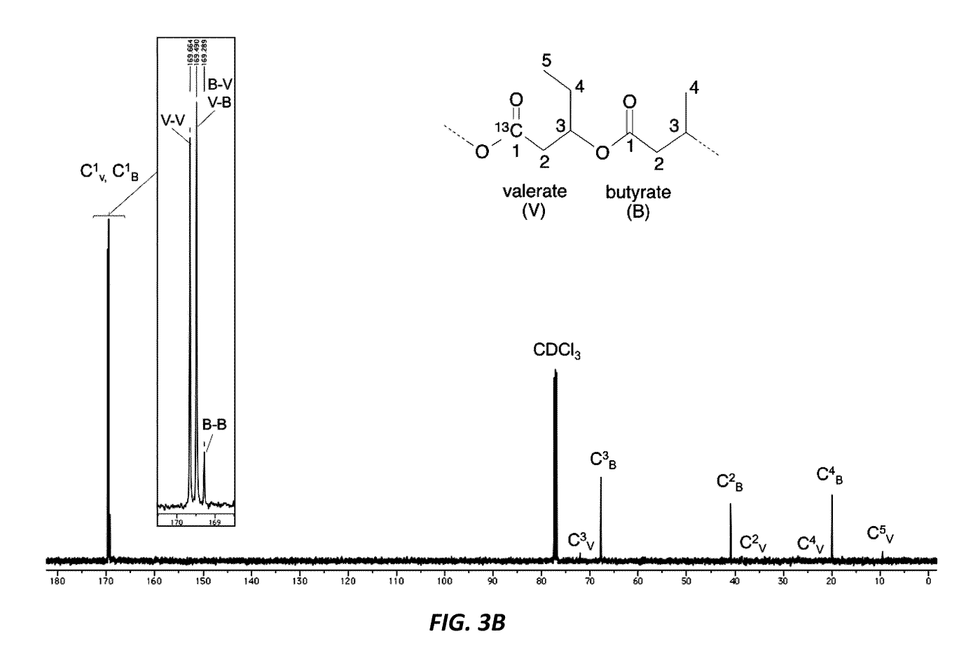

For isotopic enrichment, ¹³C-NMR spectra of PHAs made with both naturally abundant (99% ¹²C, 1% ¹³C) and isotopically labeled (99% ¹³C, 1% ¹²C on the carbonyl carbon atoms) valerate as co-substrate were recorded (FIG. 3A and FIG. 3B respectively). Both samples showed three peaks at a chemical shift (δ) of 169.3-169.7 ppm, corresponding to the carbonyl carbons in the PHAs. The presence of three carbonyl carbon peaks separated by approximately δ 0.2 ppm at this chemical shift is well-documented in PHBV copolymers, and is due to the presence of the following diads: valerate-valerate (V-V) at δ 169.7 ppm, butyrate-valerate (B-V) and valerate-butyrate (V-B) overlapping at δ 169.5 ppm, and butyrate-butyrate (B-B) at δ 169.3 ppm. Significantly, the peaks corresponding to V-V and B-V/V-V are much greater in relative intensity in the sample with [1-¹³C]valerate (FIG. 3B) than the sample with naturally abundant valerate as co-substrate (FIG. 3A), indicating that the ¹³C-carbonyl carbon atom in the [1-¹³C]valerate co-substrate has become incorporated as a ¹³C-carbonyl atom in the valerate subunit in the PHBV copolymer.

The current invention is the first innovation of a CH₄- or CH₃OH-dependent production of PHA copolymer, and the first evidence that well-known Type II obligate methanotrophs can produce copolymers. The 3HV units derived from added odd carbon fatty acids (propionate, valerate) are incorporated into PHA granules, and that the mol % 3HV in the copolymer can be adjusted by manipulating the added fatty acid concentration. Uptake of the fatty acids and incorporation of 3HV units requires oxidation of CH₄ or CH₃OH. While both propionate and valerate can be added to modify the mol % 3HV composition, valerate addition yields PHBV with a higher mol % 3HV and higher wt % PHA.

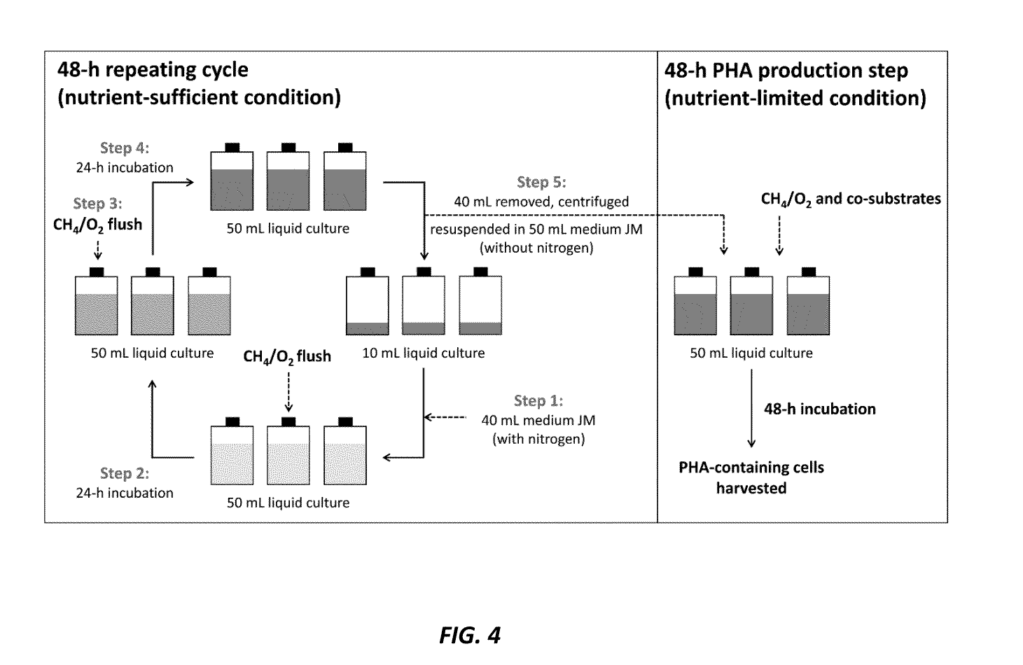

FIG. 4 shows a flow diagram of a method of producing polyhydroxyalkanoic acid (PHA)-producing biomass is provided that includes using a first bioreactor for growth of methanotrophic biomass, flushing the methanotrophic biomass with a CH₄:O₂ mixture and providing nutrients needed for sustained cell division, removing a portion of the flushed biomass, where the remainder is retained in the first bioreactor as starter biomass for continuous cycles of cell replication, transferring the removed biomass to a second bioreactor, incubating the removed biomass in the second bioreactor with a CH₄:O₂ mixture or CH₃OH:O₂ mixture in the absence of sufficient nutrients for cell replication and in the presence of a co-substrate, and harvesting PHA-containing cells from the second bioreactor.

According to one aspect of the invention, the co-substrate includes fatty acids, where the fatty acids include valerate, ¹³C-carbonyl labeled-valerate, 3-hydroxybutyrate, 4-hydroxybutyrate, or propionate.

According to another aspect of the invention, the PHA-containing cells are copolymers of PHA that include poly(3-hydroxybutyric acid-co-3-hydroxyvaleric acid) (PHBV), block-poly-3-hydroxyhexanoate (PHB-b-PHHx) or poly(3-hydroxybutyrate-co-4-hydroxybutyrate) (P3HB4HB). In one aspect, the co-substrate for the poly(3-hydroxybutyric acid-co-3-hydroxyvaleric acid) (PHBV) includes 2-pentenoate. In another aspect, the co-substrate for the block-poly-3-hydroxyhexanoate (PHB-b-PHHx) includes hexanoate. In a further aspect, the co-substrate for the poly(3-hydroxybutyrate-co-4-hydroxybutyrate) (P3HB4HB) includes 4-hydroxybutyrate and gamma butyrolactone (GBL).

Experimental procedures are disclosed herein. In one exemplary embodiment, fresh activated sludge was obtained from the aeration basin at a Regional Water Quality Control Plant. Large material was removed by filtering through a 100-μm cell strainer. The dispersed cells were centrifuged for 15 min to create a pellet. The pellet was resuspended in medium JM2 and shaken to obtain a dispersed cell suspension. Aliquots (15 mL) of the suspension were added to two serum vials containing 35 mL of medium JM2. Every 24 h for two weeks, the headspace of each bottle was flushed with a CH₄:O₂ mixture (molar ratio of 1:1.5) and amended with 0.5 mL of ammonium stock solution (1.35 M ammonium chloride; >99.8% purity). When the culture reached a final optical density (OD₆₀₀) of 1.2, it was centrifuged (3,000×g) for 15 min, and the pellet resuspended in 15 mL of medium JM2. The suspension was divided into 5-mL aliquots for inoculation of three fed-batch serum bottle cultures. Each fed-batch culture initially contained 5 mL of inoculum, 44.5 mL of medium JM2, and 0.5 mL of ammonium stock (total volume 50 mL). After a 24-h incubation period, each of the three enrichments was subject to a long-term cyclic feeding and wasting regime, with alternating pulses of CH₄ and ammonium. A repeating 48-h repeating fed-batch cycle was established enabling nearly continuous exponential growth (FIG. 4). In Step 1, all cultures with 10 mL of carry-over culture from the previous cycle received 40 mL of fresh medium (39.5 mL of medium JM2 plus 0.5 mL of ammonium stock) and were flushed for 5 min with a CH₄:O₂ mixture (molar ratio of 1:1.5). In Step 2, all cultures were incubated at 30° C. with exponential growth over a 24-h period. In Step 3, all cultures received a second 5 min headspace flush with a CH₄:O₂ mixture (molar ratio of 1:1.5). In Step 4 all cultures were incubated at 30° C. with exponential growth over a second 24-h period. Finally, in Step 5, 40 mL of liquid was quickly removed (5 min) from all cultures, completing a cycle. This fed-batch cycling was repeated more than 80 times with reproducible growth patterns.

FIG. 4 also illustrates production of PHA under nitrogen-limited growth conditions. A portion of the samples removed in Step 5 was centrifuged (3,000×g) for 15 min then suspended in fresh medium without nitrogen. The headspace of each bottle was filled with a CH₄:O₂ gas mixture (molar ratio of 1:1.5) at t=0 h and again at t=24 h. Some samples were amended with sodium valerate (>99.0% purity) to assess PHBV production. Other samples were also amended with sodium formate (60 mM). After 48 h of incubation, cells were harvested from the triplicate samples by centrifugation (3,000×g) and freeze-dried. Preserved samples were assayed for PHA content.

Turning now to the culture conditions, unless otherwise specified, all cultures were grown in medium JM. Medium JM contained the following chemicals per L of solution: 2.4 mM MgSO₄. 7H₂O, 0.26 mM CaCl₂, 3.6 mM NaHCO₃, 4.8 mM KH₂PO₄, 6.8 mM K₂HPO₄, 10.5 μM Na₂MoO₄. 2H₂O, 7 μM CuSO₄. 5H₂O, 200 μM Fe-EDTA, 530 μM Ca-EDTA, 5 mL trace metal solution, and 20 mL vitamin solution. The trace stock solution contained the following chemicals per L of solution: 500 mg FeSO₄.7H₂O, 400 mg ZnSO₄.7H₂O, 20 mg MnCl₂.7H₂O, 50 mg CoCl₂.6H₂O, 10 mg NiCl₂.6H₂O, 15 mg H₃BO₃, 250 mg EDTA. The vitamin stock solution contained the following chemicals per L of solution: 2.0 mg biotin, 2.0 mg folic acid, 5.0 mg thiamine.HCl, 5.0 mg calcium pantothenate, 0.1 mg vitamin B 12, 5.0 mg riboflavin, and 5.0 mg nicotinamide.

All cultures were incubated in 160 mL serum bottles capped with butyl-rubber stoppers and crimp-sealed under 1:1.5 CH₄:O₂ 110 mL headspace (>99% purity). Bottles were incubated horizontally on orbital shaker tables at 150 rpm. The incubation temperature was 30° C.

Fifty-millimeter cultures were grown to final optical densities (OD₆₀₀) of 0.8 to 1.2 then centrifuged (3,000×g) for 15 min. The pellets were resuspended in 30 mL of JM medium to create the inoculum for triplicate 160 mL serum bottle cultures. Each culture received 10 mL inoculum plus 40 mL of fresh medium (39.5 mL of medium JM plus 0.5 mL of 1.35 M ammonium stock) and was flushed for 5 min with a CH₄/O₂ mixture (molar ratio of 1:1.5). After growth at 30° C. for 24 h, the headspace in each culture was again flushed for 5 min with the CH₄/O₂ mixture then incubated at 30° C. for a second 24 h period of exponential growth.

After 48 h, all cultures were harvested and subjected to nitrogen-limiting conditions. Triplicate samples were centrifuged (3,000×g) for 15 min and suspended in fresh medium without nitrogen. The headspace of each bottle was flushed with the CH₄:O₂ gas mixture at t=0 h and t=24 h. In some cases, the medium was amended with 100 mg/L sodium 3-hydroxybutyrate (3HB, >99.0% purity), 100 mg/L sodium propionate (>99.0% purity), or 0-2000 mg/L sodium valerate (>99.0% purity). After 48 h of incubation, cells were harvested by centrifugation (3,000×g) and freeze-dried. Preserved samples were assayed for PHA content. To confirm valerate incorporation in PHA granules, [1-¹³C]valerate ([1-¹³C]valeric acid) was used as a co-substrate.

To test use of alternative carbon sources, cultures were incubated in presence of 3HB (100-2000 mg/L), propionate (100-2000 mg/L) or valerate (100-2000 mg/L), but without CH₄. Control cultures without any added substrate were also prepared. OD₆₀₀ values were measured for 45 d.

To test culture purity, biomass was removed after the 48-h period of exponential growth. Genomic DNA was extracted using the FastDNA SPIN Kit for Soil (MP Biomedicals, Santa Ana, Calif., USA), as per the manufacture's protocol. Bacterial 16S rRNA was amplified using the bacterial primers BAC-8F (5′-AGAGTTTGATCCTGGCTCAG-3′) and BAC-1492R (5′-CGGCTACCTTGTTACGACTT-3′). A Polymerase chain reaction (PCR) was performed using Accuprime Taq DNA Polymerase System and the following thermocycling steps: (i) 94° C. for 5 min; (ii) 30 cycles consisting of 94° C. for 30 s, 55° C. for 30 s, 68° C. for 80 s; and (iii) an extension at 68° C. for 10 min. Amplicon presence and quality of PCR reaction were verified via 1.5% agarose gel electrophoresis.

PCR products were purified using QIAquick PCR Purification Kit, then cloned using pGEM-T Easy Vector System with JM109 competent Escherichia coli cells per the manufacture's protocol. Randomly selected clones were sequenced generating 120 near-full length 16S rRNA gene sequences. Retrieved DNA sequences were compared with reference sequences using Basic Local Alignment Search Tool (BLAST).

To determine PHA weight percent and monomer composition, between 5 and 10 mg of freeze-dried biomass were weighed then transferred to 12 mL glass vials. Each vial was amended with 2 mL of methanol containing sulfuric acid (3%, vol/vol) and benzoic acid (0.25 mg/mL methanol), supplemented with 2 mL of chloroform, and sealed with a Teflon-lined plastic cap. All vials were shaken then heated at 95-100° C. for 3.5 h. After cooling to room temperature, 1 mL of deionized water was added to create an aqueous phase separated from the chloroform organic phase. The reaction cocktail was mixed on a vortex mixer for 30 s then allowed to partition until phase separation was complete. The organic phase was then sampled by syringe and analyzed using a GC equipped with an column containing 5% phenyl-methylpolysiloxane and a flame ionization detector. DL-hydroxybutyric acid sodium salt and PHBV with 3HV fractions of 5 mol %, 8 mol % and 12 mol % were used to prepare external calibration curves. The PHA content (wt %, w_{PHA}/w_{CDW}) of the samples and 3HV fraction of the PHAs (mol %) were calculated by normalizing to initial dry mass.

Regarding purification, PHA granules were extracted from the cells by suspending 500 mg of freeze-dried cell material in 50 mL Milli-Q water, adding 400 mg of sodium dodecyl sulfate (>99.0% purity and 360 mg of EDTA, followed by heating to 60° C. for 60 min to induce cell lysis. The solution was centrifuged (3,000×g) for 15 min, and the pellet washed three times with deionized water. To purify the PHA, pellets were washed with a 50-mL sodium hypochlorite (bleach) solution (Clorox 6.15%), incubated at 30° C. with continuous stirring for 60 min, then centrifuged (3,000×g) for 15 min. Sample pellets were washed and re-centrifuged three times with deionized water.

Molecular weights of PHAs were evaluated using gel permeation chromatography (GPC). Sample pellets dissolved in chloroform at a concentration of 5 mg/mL for 90 min at 60° C. were filtered through a 0.2-μm PTFE filter, then analyzed with an ultra fast liquid chromatography system equipped with a refraction index detector. The GPC was equipped with a Jordi Gel DVB guard column (500 Å) and Jordi Gel DVB analytical columns (10⁵ Å). The temperature of the columns was maintained at 40° C., and the flow rate of the mobile phase (chloroform) was 1 mL min⁻¹. Molecular weights were calibrated with polystyrene standards.

Peak melting temperatures (Tₘ) and onset glass transition temperatures (T⁰_{g}) of PHAs were evaluated using TA Q2000 differential scanning calorimetry. Thermal data were collected under a nitrogen flow of 10 mL min⁻¹. About 5 mg of melt-quenched PHA samples encapsulated in aluminum pans were heated from −40° C. to 200° C. at a rate of 10° C. min⁻¹. The peak melting temperatures were determined from the position of the endothermic peaks.

For Nuclear Magnetic Resonance (NMR), ¹³C (125 MHz, 1048 scans, delay time (d1)=0.5 s) NMR spectra of PHAs were recorded at room temperature on a 500 MHz spectrometer, with shifts reported in parts per million downfield from tetramethylsilane and referenced to the residual chloroform solvent peak (77.16 ppm). Samples were prepared by adding 3 mg of the PHA to 0.7 mL deuterated chloroform (CDCl₃), with gentle heating until the PHA had fully dissolved.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. All such variations are considered to be within the scope and spirit of the present invention as defined by the following claims and their legal equivalents.

What is claimed:

1. A method of producing polyhydroxyalkanoic acid (PHA)-producing biomass, comprising:
a. using a first bioreactor for growth of methanotrophic biomass;
b. flushing the methanotrophic biomass with a CH₄:O₂ mixture and providing nutrients needed for sustained cell division;
c. removing a portion of said flushed biomass, wherein the remainder is retained in said first bioreactor as starter biomass for continuous cycles of cell replication;
d. transferring said removed biomass to a second bioreactor;
e. incubating said removed biomass in said second bioreactor with said CH₄:O₂ mixture or a CH₃OH:O₂ mixture in the absence of sufficient nutrients for cell replication and in the presence of a co-substrate; and
f. harvesting PHA-containing cells from the second bioreactor.

2. The method of claim 1, wherein said co-substrate comprises fatty acids, wherein said fatty acids are selected from the group consisting of valerate, ¹³C-carbonyl labeled-valerate, 3-hydroxybutyrate, 4-hydroxybutyrate, and propionate.

3. The method according to claim 1, wherein said PHA-containing cells comprise copolymers of PHA comprising poly(3-hydroxybutyric acid-co-3-hydroxyvaleric acid) (PHBV), block-poly-3-hydroxyhexanoate (PHB-b-PHHx) or poly(3-hydroxybutyrate-co-4-hydroxybutyrate) (P3HB4HB).

4. The method according to claim 3, wherein said co-substrate for said poly(3-hydroxybutyric acid-co-3-hydroxyvaleric acid) (PHBV) comprises 2-pentenoate.

5. The method according to claim 3, wherein said co-substrate for said block-poly-3-hydroxyhexanoate (PHB-b-PHHx) comprises hexanoate.

6. The method according to claim 3, wherein said co-substrate for said poly(3-hydroxybutyrate-co-4-hydroxybutyrate) (P3HB4HB) comprises 4-hydroxybutyrate and gamma butyrolactone (GBL).
